(19) United States
(12) Patent Application Publication
(10) Pub. No.: US 2013/0231458 A1
(43) Pub. Date: Sep. 5, 2013

(54) POLYPEPTIDE FOR PREVENTING AND TREATING CELL HYPERPLASIA

(71) Applicant: Hueih-Min CHEN, Hsinchu City (TW)

(72) Inventor: Hueih-Min CHEN, Hsinchu City (TW)

(21) Appl. No.: 13/312,039

(22) Filed: Dec. 6, 2011

(51) Int. Cl. C07K 14/00

(52) U.S. Cl. 530/324

(57) ABSTRACT

A polypeptide for preventing and treating cell hyperplasia is applied to preventing potential objects from cell hyperplasia and treating objects having cell hyperplasia. The polypeptide comprises an amino acid sequence listed as SEQ ID NO: 1. The amino acid sequence can effectively inhibit growth of lung cancers. The polypeptide would not cause significant evil reactions or obvious side effects even though it is applied at a high dose (50 mg/Kg) and a high frequency (5 times per week).

### FIELD OF THE INVENTION

The present invention relates to a polypeptide, particularly to a polypeptide for preventing and treating cell hyperplasia.

### BACKGROUND OF THE INVENTION

The conventional cancer prevention technology includes the carcinogenicity inspection technologies and the anti-cancer vaccine technologies. The carcinogenicity inspection technologies include (A) the intrinsic carcinogenicity inspection technology, (B) the extrinsic carcinogenicity inspection technology, and (C) the biological carcinogenicity inspection technology. Few intrinsic carcinogenicity-related factors can be used in inspection of intrinsic carcinogenicity, including age, heredity, ethnicity, sexuality, etc. The intrinsic carcinogenicity inspection may be applied to the high-risk population having a hereditary carcinogenic factor, such as the BRCA-1 gene or the BRCA-2 gene. According to the inspection result and the family morbidity, the region likely to have cancer in the future is resected preventively. The extrinsic carcinogenic factors include chemical carcinogenic factors, physical carcinogenic factors and environmental carcinogenic factors. The physical carcinogenic factors include ultraviolet light and radioactive rays. The chemical carcinogenic factors include benzpyrene in tobacco, colorants, flavoring agents and preservatives in food. The environmental carcinogenic factors include organic chemical wastes, tar residues, aromatic cosmetic dyes, agrochemicals, pesticides, environmental hormones, arsenic, and mercury. Some biological agents and viruses closely correlate with cancer. The biological carcinogenic factors include aflatoxin, HBV (Hepatitis B Virus) and HPV (Human Papilloma Virus). HBV has a direct relationship with hepatoma. HPV has a direct relationship with cervical cancer. According to the timing that the chemoprevention is practiced, the chemopreventions may be classified into the primary chemoprevention for the initial stage of cancer and the secondary chemoprevention for the patients having suffered from cancer and having be treated appropriately. For example, Vit A retinyl palmitate and ADT (5-[p-Methoxyphenyl]-1,2-dithiole-3-thione) are used to treat lung cancer; retinoic acid is used to treat head-neck cancer; Tamoxifen is used to treat breast cancer; Sulindac is used to treat colon cancer. Self-carefulness in public health and sexual safety is very useful in preventing cancers induced by biological agents. Besides, vaccination can effectively promote the immunity against the biologically-induced cancers. For example, HPV vaccine can protect a specified gender from HPV infection; HBV vaccine can interrupt the process of hepatitis B-hepatic cirrhosis-hepatic cancer.

### SUMMARY OF THE INVENTION

The primary objective of the present invention is to provide chemoprevention and chemotherapy for cell hyperplasia, wherein the chemoprevention of cell hyperplasia in the present invention is referred to using a natural, synthesized or biochemically synthesized drug to reverse, suppress, or prevent oncogenesis (Refer to Anne S. Tsao, MD; Edward S. Kim, MD; Waun Ki Hong, MD; CA Cancer J Clin 2004; 54: 150-180).

To achieve the abovementioned objective, the present invention proposes a polypeptide for preventing and treating cell hyperplasia, which is an amino acid sequence listed as SEQ ID NO: 1.

The polypeptide of the present invention contains the amino acid sequence listed as SEQ ID NO: 2 and the amino acid sequence listed as SEQ ID NO: 3, which may be mathematically expressed by AM1+AM2+AM4+AM3, wherein AM1, AM2 and AM3 have the same amino acid sequence listed as SEQ ID NO: 2 and AM4 has the amino acid sequence listed as SEQ ID NO: 3. In the polypeptide of the present invention, three amino acid fragments of AM 4 are used to connect (AM1+AM2) and AM3 (Note: AM1, AM2 and AM3 may respectively have different compositions). The present invention adopts AM1, AM2 and AM3 of an identical composition so as to form a section able to bond with the compounds on the surface of cancer cells (most of them are heparin sulfates). The section is in form of [EKKWKV] that matches a mode of [XBBXBX] for bonding with heparin sulfates, wherein B denotes a basic amino acid and X denotes an arbitrary one of other amino acids (Refer to I. Capila and R. J. Linhardt, Heparin-protein interactions, Angew. Chem. Int. Ed Engl. 2002; 41: 391). Such a design makes the polypeptide of the present invention have greater affinity and increases the total charges to as high as +12. The high positive charge number greatly increases the capability of the polypeptide to bind with the negatively-charged surface of cancer cells. The polypeptide is normally structureless in aqueous solutions, i.e. in form of random coils. Once the polypeptide exists in a polar environment, such as a cell membrane, it form a 3D structure of two α-helixes twisted at AGP. The U-shape structure makes the polypeptide be easily introduced into cell membranes to dynamically react with different levels of cell surface and rapidly disintegrate cells.

The polypeptide of the present invention is highly specific to abnormally proliferative cells and hydrolyzes much more tumor cells than normal cells. Besides, the polypeptide of the present invention would not cause significant evil reactions or obvious side effects within the dose range for chemoprevention or chemotherapy (including a high dose).

Below, the embodiments are described in detail to make easily understood the objectives, characteristics and advantages of the hyper-proliferative chemoprevention and chemotherapy polypeptide of the present invention.

### DETAILED DESCRIPTION OF THE PREFERRED EMBODIMENTS

Based on the experimental results obtained from the following embodiments, the present invention discloses an anti-hyper-proliferation polypeptide containing an amino acid sequence listed as SEQ ID NO: 1. The polypeptide of the present invention applies to preventing and treating cellular proliferation disorders and may be mathematically expressed by AM1+AM2+AM4+AM3, wherein AM1, AM2 and AM3 has the same amino acid sequence listed as SEQ ID NO: 2 and AM4 has the amino acid sequence listed as SEQ ID NO: 3. The polypeptide of the present invention is used as a preventive medicine to reduce the morbidity of the high risk groups of cell hyperplasia and a therapeutic medicine to inhibit relapse of cell hyperplasia after the patients have been treated. The polypeptide of the present invention is delivered into a human body via subcutaneous injection or intravenous injection. The high risk groups of cell hyperplasia include tumor patients, people living in high air pollution areas, smokers, passive smokers, etc. The diseases of cell hyperplasia include cancers, such as lung cancers. The polypeptide of the present invention has superior cancer-prevention and cancer-therapy effects. The polypeptide of the present invention would not cause significant evil reactions or obvious side effects even though it is applied at a high dose (50 mg/Kg) and a high frequency (5 times per week).

### Embodiment I

The polypeptide of the present invention is fabricated with a solid-phase peptide synthesis method, which was invented by Prof. R. B. Merrifield (Nobel Laureate 1984), Rockefeller University, USA. The solid-phase peptide synthesis method uses a coupling effect to covalently bond activated amino acids to a solid matrix resin, wherein the amino acid at the first position of the sequence is bonded to the N terminal (the NH₂ terminal), and next the amino acids Trp-2, Lys-3, Val-4, Phe-5, Lys-6, Lys-7, Ile-8, Glu-9, Lys-10, . . . , Glu-32 are sequentially bonded to the solid matrix resin until the last amino acid Lys-33 is bonded to form a peptide, and then the synthesized peptide is flushed out from the resin to obtain the desired peptide. There are two approaches to realize the solid-phase peptide synthesis method: the Boc approach and the Fmoc approach. The two approaches are similar but different in the functional groups that protect the N terminal of the amino acids. The peptides having a length of 2-50 amino acids can be synthesized with the abovementioned method, and the yield thereof may amount to grams, kilograms or even tons. The polypeptide of the present invention have a length of 33 amino acids, and the purity thereof may achieve as high as over 95% via the abovementioned method.

### Embodiment II

An in-vitro experiment uses lung cancer spheroids to test the cancer-inhibiting effect of the polypeptide, wherein the polypeptide of the present invention is applied before and after the formation of the lung cancer (NCI-H460) agglomerates. The experiment is undertaken according to a paper by Timmins et al, Angiogenesis 7: 97-103, 2004: cultivate cells in culture dishes until 70-80% area of the culture dishes is covered by cells; use Trypsin to separate cells from the culture dishes; centrifugally process the collected cells, and count the cells in the precipitate; drip a 20 μl droplet containing 500 cells on the top cover of each culture dish to suspend the cells stilly and let gravity attract the cells to agglomerate and form cancer spheroids. The polypeptide is applied to the cancer cells before, during and after suspension to examine the effect thereof. In the case that the polypeptide is not applied to the cancer cells from start to end, the cancer cells grow to form agglomerates. In the case that the polypeptide is applied to the cancer cells during growth, the cancer cells grow to form smaller agglomerates. In the case that the polypeptide is applied to the cancer cells before suspension, the cancer cells do not form agglomerates. The experiment proves that the polypeptide has superior capability of preventing the lung cancer cells from growing into agglomerates.

### Embodiment III

The polypeptide of the present invention is delivered to the target via blood circulation (such as via subcutaneous injection or intravenous injection). An erythrocyte poisoning experiment is undertaken to examine the erythrocyte toxicity of the polypeptide of the present invention. Refer to a paper by Wu et al., Peptides 30, 839-848, 2009, for the experimental result. In the case that the concentration of the polypeptide is lower than 25 μM, almost none erythrocyte is damaged. In the case that the concentration of the polypeptide is increased to as high as 200 μM, only about 20% of erythrocytes are damaged. The erythrocyte toxicity of the polypeptide of the present invention is similar to that of the clinic anti-cancer medicine Doxorubicin. The other types of peptides, such as melittin, may damage about 90% of erythrocytes at a very low concentration (lower than 5 μM). Therefore, the polypeptide of the present invention has very low erythrocyte toxicity.

The polypeptide of the present invention can effectively inhibit the in-vitro lung cancer cells but would not obviously poison the normal cells (including normal blood cells). Hence, an animal experiment is undertaken in a xenograft model to examine the chemoprevention effect of the polypeptide of the present invention on the in-vivo lung cancer cells, wherein the polypeptide is delivered to immunodeficient nude mice via subcutaneous injection.

### Experimental Process

The polypeptide of the present invention is preventively injected into the rear parts of the necks of four nude mice (nude mice—NU/NU) for a week (5 times per week at a dose of 50 mg/Kg). Next, 1×10⁶ lung cancer cells are injected into the hypodermic tissue of the right side of the abdomen of each nude mouse on Day 1. The abovementioned four nude mice function as the medicine test group. Next, the polypeptide is delivered to each nude mouse of the medicine test group at a dose of 50 mg/Kg 5 times per week from Day 1 to Day 43. Meanwhile, the weight and cancer size of each nude mouse of the medicine test group is recorded. The negative control group also has four nude mice. Normal saline having a pH of 7.2-7.4 is subcutaneously injected into the four nude mice of the negative control group for a week (5 times per week). Next, 1×10⁶ lung cancer cells are injected into the hypodermic tissue of the right side of the abdomen of each nude mouse of the negative control group on Day 1. Next, normal saline is delivered to each nude mouse of the negative control group 5 times per week from Day 1 to Day 43. Meanwhile, the weight and cancer size of each nude mouse of the negative control group is recorded. The positive control group also has four nude mice. 1×10⁶ lung cancer cells are injected into the hypodermic tissue of the right side of the abdomen of each nude mouse of the positive control group on Day 1. Next, a small-molecule anti-cancer drug Doxorubicin is intravenously injected into a region near the abdomen of each nude mouse of the positive control group once per week at a dose of 5 mg/Kg from Day 1 to Day 43 (refer to F Pastorino et. al., Clin. Cancer Res. 14(22): 7320-7329, 2008). Meanwhile, the weight and cancer size of each nude mouse of the positive control group is recorded. Different from the abovementioned two groups, medicine is not applied to the positive control group before Day 1. The medicine test group, negative control group and positive group are observed until Day 43. The mice of the three groups are dissected for examining and recording the weight of the tumor tissue on Day 43.

### Experimental Results

Refer to Table.1 and Table.2 respectively recording weights and tumor volumes of nude mice.

The weights (shown in Table.1) and motilities of the three groups do not vary significantly during the experiment. However, the tumor volumes (shown in Table.2) of the three groups diverge obviously in the final stage of the experiment. After the lung cancer cells had been injected into the mice for 22 days, hypodermic phymata begin to appear. The tumor volumes of the negative control group (saline) and the positive control group (Doxorubicin) increase with the elapsed time. However, the medicine test group (polypeptide) performs differently. The ratio of the average tumor volumes of the positive control group (Doxorubicin) and the negative control group (saline) is 52.0% at best on Day 40. The ratio of average tumor volumes of the medicine test group (polypeptide) and the negative control group (saline) is 13.89% at best on Day 36. In the end of the experiment (Day 43), the ratio of the average tumor volumes of the positive control group (Doxorubicin) and the negative control group (saline) is 83.05%; the ratio of average tumor volumes of the medicine test group (polypeptide) and the negative control group (saline) is 19.30%. Therefore, the experiment proves that the mode involving preventively applying the polypeptide to the animals for one week can effectively inhibit growth of the xenograft lung cancers.

In conclusion, the polypeptide of the present invention has high specificity to cancer cells and can effectively hydrolyze cancer cells. Therefore, the polypeptide of the present invention is very useful in chemoprevention and chemotherapy of cell hyperplasia. Further, the polypeptide of the present invention would cause significant evil reactions or obvious side effects even though it is applied at a high dose (50 mg/Kg) and a high frequency (5 times per week).

It is possible for each technology disclosed in the present invention to be integrated with another technology or replaced with a similar or equivalent technology. According to the technologies disclosed in the present, the persons skilled in the art should be able to make modifications or variations of the present invention without departing from the spirit of the present invention. However, any modification or variation according to the spirit of the present invention is to be also included within the scope of the present invention.

TABLE 1
Weights of Nude Mice (Unit: gram)

| | Date | | | | | | | | | | | | |
|---|---|---|---|---|---|---|---|---|---|---|---|---|---|
| Drug | D1 | D5 | D8 | D12 | D15 | D19 | D22 | D26 | D29 | D33 | D36 | D40 | D43 |
| Dox | 25.6 | 26.5 | 27.8 | 28.2 | 28.8 | 30.4 | 30.0 | 30.4 | 30.0 | 31.5 | 32.1 | 32.5 | 32.8 |
| Saline | 25.3 | 25.1 | 26.4 | 27.2 | 28.4 | 29.6 | 29.2 | 29.6 | 29.2 | 30.8 | 31.1 | 31.7 | 32.8 |
| Polypeptide | 25.5 | 25.5 | 26.4 | 27.0 | 28.0 | 29.6 | 29.1 | 29.6 | 29.1 | 30.3 | 30.6 | 31.1 | 31.5 |

Dox: positive control group;
Saline: negative control group;
polypeptide: medicine test group

TABLE 2
Tumor Volumes of Nude Mice (Unit: mm³)

| | date | | | | | |
|---|---|---|---|---|---|---|
| drug | D22 | D29 | D33 | D36 | D40 | D43 |
| Dox | 57.8 | 373.8 | 553.5 | 632.6 | 884.5 | 1308.9 |
| Saline | 81.7 | 488.8 | 828.1 | 1555.0 | 1800.0 | 2209.6 |
| polypeptide | 12.9 | 70.6 | 137.3 | 166.8 | 279.3 | 451.2 |

Dox: positive control group;
Saline: negative control group;
polypeptide: medicine test group

1. A polypeptide for preventing and treating cell hyperplasia, which applies to preventing potential objects from cell hyperplasia and treating objects having cell hyperplasia, the polypeptide comprising an amino acid sequence listed as SEQ ID NO: 1.

2. The polypeptide for preventing and treating cell hyperplasia according to claim 1, wherein the polypeptide contains a U-shape 3D structure having two α-helixes twisted at three amino acids AGP, wherein the U-shape 3D structure is able to prevent and treat cell hyperplasia.

3. The polypeptide for preventing and treating cell hyperplasia according to claim 1, wherein the polypeptide is applied to preventively treating high-risk groups of cell hyperplasia.

4. The polypeptide for preventing and treating cell hyperplasia according to claim 1, wherein the polypeptide is applied to preventing relapse of cell hyperplasia that have been treated beforehand.

5. The polypeptide for preventing and treating cell hyperplasia according to claim 1, wherein the objects are human bodies.

6. The polypeptide for preventing and treating cell hyperplasia according to claim 5, wherein the polypeptide is delivered into human bodies via subcutaneous injection or intravenous injection.

7. The polypeptide for preventing and treating cell hyperplasia according to claim 5, wherein the polypeptide is applied to human bodies at a dose of 50 mg/Kg and a frequency of 5 times per week.

8. The polypeptide for preventing and treating cell hyperplasia according to claim 1, wherein the cell hyperplasia include cancers.

9. The polypeptide for preventing and treating cell hyperplasia according to claim 8, wherein the cancers include lung cancers.

10. The polypeptide for preventing and treating cell hyperplasia according to claim 1, wherein the potential objects of cell hyperplasia include people living in a high air pollution area, smokers, and passive smokers and the objects having cell hyperplasia include persons suffering from cancer.


SEQUENCE LISTING (3 sequences)

SEQ ID NO: 1 - protein, 33 aa, Artificial Sequence
  Description of Artificial Sequence Synthetic peptide
KWKVFKKIEKKWKVFKKIEKAGPKWKVFKKIEK

SEQ ID NO: 2 - protein, 10 aa, Artificial Sequence
  Description of Artificial Sequence Synthetic peptide
KWKVFKKIEK

SEQ ID NO: 3 - protein, 3 aa, Artificial Sequence
  Description of Artificial Sequence Synthetic peptide
AGP